(19) United States
(12) Patent Application Publication
(10) Pub. No.: US 2013/0131460 A1
(43) Pub. Date: May 23, 2013

(54) PHYSILOGICAL SIGNAL COLLECTION APPARATUS AND PERFORMANCE MONITORING APPARATUS INCORPORATING SAME

(71) Applicant: Paul Anthony Yuen, 11-A Kwai Chung (HK)

(72) Inventor: Paul Anthony Yuen, 11-A Kwai Chung (HK)

(73) Assignee: DAYTON TECHNOLOGIES LIMITED, 11-A Kwai Chung, New Territories (CN)

(21) Appl. No.: 13/520,862

(22) PCT Filed: Jan. 10, 2011
(86) PCT No.: PCT/IB11/50078

§ 371 Date: Jul. 6, 2012

(30) Foreign Application Priority Data
Jan. 8, 2010 (HK) 10100226.4

(51) Int. Cl. A61B 5/0408; A61B 5/00; A61B 5/11; A61B 5/04

(52) U.S. Cl. CPC A61B 5/04085; A61B 5/04; A61B 5/6831; A61B 5/04012; A61B 5/6814; A61B 5/6824; A61B 5/6828; A61B 5/6823; A61B 5/11
USPC 600/301; 600/382; 600/383; 600/384

(57) ABSTRACT

A physiological signal collection electrode comprises a signal collection pad having a skin contact portion, a signal output pad and an elongate bridging portion interconnecting the signal collection pad and the signal output pad. The signal collection pad, the signal output portion and the bridging portion are integrally moulded of a flexible, conductive and resilient material. The width of the bridging portion is substantially smaller than that of the skin contact portion. A narrowed bridging portion operates to concentrate collected physiological signals collected by the skin contact portion before the signals are output to the signal output pad. An elongate bridging portion reduces skin covering area for better wearer comfort as well as providing better resiliency to the electrode when the bridging portion is extended.

### FIELD OF THE INVENTION

The present invention relates to physiological signal collection apparatus, and more particularly to chest-worn physiological signal collection apparatus. This invention also relates to performance monitoring apparatus comprising a physiological signal collector and a signal processing device for processing collected physiological signals. More specifically, although not solely limited thereto, the present invention also relates to chest-worn swim monitors.

### BACKGROUND OF THE INVENTION

Physiological signal collectors are useful for collecting physiological data of a person during physical exercises, during medical examination or during everyday life. Typical physiological signals which are commonly collected for processing and analyzing include ECG, heart rate, blood pressure, blood oxygen content, body temperature. The collected signals are typically processed and converted into data which provide information on the state of health, physical fitness, or physical performance of a person.

A chest strap is a known type of physiological signal collectors adapted to be chest-worn by a person for collecting physiological signals during physical exercises or activities. Physiological signals commonly collected by a chest strap include, for example, heart-rate, ECG pulses, skin conductivity, infra-red absorption or other electrical or opto-electrical signals measureable from the skin of a person.

A typical chest strap usually includes a plurality of electrodes or sensors which is mounted on a flexible plastic chest strap in a spaced apart manner to collect weak physiological signals in electrical or optical form from a human body for differential signal processing. Each electrode typically comprises a signal reception pad having a signal reception surface with a surface area large enough to collect signals which are strong enough for processing by a signal processing device, such as a heart rate signal processor.

The flexible plastic strap is usually a pre-assembled strap loop which comprises a length adjustment or tension adjustment arrangement to tighten the strap against the body of a person, usually the chest, during use to provide electrical contact between the electrodes and the skin of a user. The strap is usually made of soft plastics to provide flexibility for body wearing and electrical insulation between the electrodes. The tensions adjustment arrangement typically comprises a length of soft plastic strap portion which runs around buckle or clasp arrangements.

However, such chest-type physiological data collectors are uncomfortable and difficult to wear and adjust. Also, it is noted that known chest straps do not operate well enough for wet applications, for example swimming or water sports.

Therefore, it would be advantageous to provide improvement chest straps to mitigate shortcoming aspects of conventional chest straps.

### SUMMARY OF THE INVENTION

According to the present invention, there is provided a physiological signal collection electrode comprising a signal collection pad having a skin contact portion, a signal output pad, and an elongate bridging portion interconnecting the signal collection pad and the signal output pad, wherein the signal collection pad, the signal output portion and the bridging portion are integrally moulded of a flexible, conductive and resilient material; characterized in that the width of the bridging portion is smaller than that of the skin contact portion.

A narrowed bridging portion operates to concentrate collected physiological signals collected by the skin contact portion before the signals are output to the signal output pad. An elongate bridging portion reduces skin covering area for better wearer comfort as well as providing better resiliency to the electrode when the bridging portion is extended. The signal collection pad coupled with the narrowed bridging portion makes the electrode more flexible and is therefore particularly suitable for collecting human ECG signals, for example, from rib cage regions of a person.

To mitigate spurious signals reception of the bridging portion, the bridging portion may be covered by a flexible, resilient and waterproof insulating material such as rubber, silicone rubber or soft plastics. The insulating material is preferably over-moulded on the electrode for better shielding against spurious signals.

The skin contact portion may elevate from the signal collection pad and may be surrounded by an insulating material. The surface of the insulating material is preferably flush with the surface of the skin contact portion, thereby making a continuous flush surface for better wear comfort while maintaining a well defined signal collection area.

The signal output pad may include a signal output terminal to output the collected physiological signal for external processing.

The effective resistance of the electrode measured between the skin contact portion and the signal output terminal is less than 1 kΩ, preferably less than 500Ω, and more preferably less than 400Ω. It has been found that such a low resistance is beneficial for in or under water applications. Accordingly, the electrode may be configured as an ECG signal collector for collecting ECG signals during swimming.

The skin contact surface may be flexible and elongate with an effective skin contact surface of between 7.5 cm² to 20 cm².

The skin contact portion may be between 1.5-2.5 cm wide and 5-8 cm long.

The narrowed bridging portion may be between 6 and 10 cm long and about half or less than half of the width of the skin contact portion.

The flexible, conductive and resilient material may be selected form carbonized rubber, carbonized fabrics, Nickel copper plated polyester, silver nylon mesh, cotton silver bamboo fiber, or other conductive fabrics such as metalized fabrics or the like.

According to another aspect of the present invention, there is provided an ECG signal collection strap comprising first and second ECG signal collection electrodes which are respectively mounted at first and second free longitudinal ends of an insulating strap, wherein each signal collection electrode comprises a signal output terminal and a signal collection pad having a skin contact surface which is adapted for collecting ECG signals from the skin of a user, characterized in that, the ECG signal collection electrode is an electrode of the present invention.

According to yet another aspect of the present invention, there is provided an ECG signal collection strap comprising first and second ECG signal collection electrodes which are respectively mounted at first and second free longitudinal ends of an insulating strap, wherein each signal collection electrode comprises a signal output terminal and a signal collection pad having a skin contact surface which is adapted for collecting ECG signals from the skin of a user, characterized in that, first and second mechanical fastening parts are provided respectively at the first and second longitudinal ends of the strap for converting the longitudinal strap into a strap loop for wearing on the body of a user, and the first and second mechanical fastening parts are arranged such that the strap will urge the skin contact surfaces of the first and second electrodes to abut skin of the user to make electrical contacts and to collect physiological signals when the strap loop is worn on the body of the user with the fastening parts engaged.

Such an arrangement facilitates front wearing of the strap as a user could engage the fastening parts at the front of the chest.

The signal output terminal comprises a mechanical mating portion of a conductive mechanical fastening part which is adapted for engagement with a complementary mechanical mating portion of a counterpart mechanical fastening part. A signal output terminal which is also a mechanical portion of a conductive mechanical fastening part means added user convenience because a user only needs to make a single connection step to achieve both electrical and mechanical connection.

The first and second mechanical fastening parts of the signal collection strap may be adapted for mechanical engagement with a portable ECG signal processing device, and to form a complete strap loop upon engagement with the portable ECG signal processing device, and wherein the strap is arranged such that ECG signals collected by the electrodes are delivered to the portable ECG signal processing device via the mechanical fastening parts. The portable ECG signal processing device is useful for ECG signal processing as well as serves as a latching bridging for converting the strap into a strap loop.

The signal output terminal may also a conductive mating portion of a snap fastener part. This facilitates snap fastening for making both electrical and mechanical connections for added convenience.

The insulating strap may be elastic and its length may be adjustable. The insulating strap may be made of air and/or moisture permeable elastomeric fabric such as elastomeric polyamide or swimwear fabric such as Nylon, Lycra®, polyester, or the like. Anti-slid texture may be formed on portions of the insulating strap which are in contact with user skin during use.

The bridging portion of each of the electrodes may be embedded within the insulated strap. The signal collection pad of each of the electrodes may be bonded on the insulated strap.

The mechanical fastening components may be arranged to convert the longitudinal tensioning strap into a tensioning strap loop The conductive parts of the mechanical fastening components may form part of the signal output terminals and/or vice versa.

The swim motion detector and the ECG signal processing device may be formed as a module and may be housed within a common housing, and the ECG signal processing device has first and second signal input terminals. The ECG signal processing device may be arranged to obtain ECG signals from the strap via the signal output terminals of the strap.

The swim motion detector may comprise a 3-axis accelerometer which is arranged to collect swim motion data.

The effective signal path resistance between the skin contact portion of the electrode and a corresponding output terminal may be less than that of the swim water signal path between the skin in contact with the electrode and the corresponding output terminal.

The monitor is adapted for use in sea water, chlorinated water or other ionized water, and the effective signal path resistance of each of the electrodes is below 800 ohms, preferably below 500 ohms.

Each of the first and second terminals of the ECG processing device may be formed as a mating portion of a mechanical fastener part, and each of the output terminals of the strap is formed into a complementary mating portion of a counterpart mechanical fastener part.

The physiological signal processing module may connect with the signal collection strap to form a complete strap loop when they are mechanically engaged.

The strap loop is a body strap such as a chest strap, head strap, arm strap, thigh strap, or the like.

### BRIEF DESCRIPTION OF THE DRAWINGS

Embodiments of the present invention will be explained below by way of example and with reference to the accompanying drawings, in which:

FIG. 1 is a perspective view of a chest strap illustrating a first embodiment of the present invention,

FIGS. 2 and 3 respectively show the front and rear views of the chest strap of FIG. 1,

FIG. 4 shows the strap of FIGS. 1-3 when fastened with an ECG processing apparatus,

FIG. 4A shows the strap of FIG. 4 when the ECG processing apparatus partly detached from the chest strap,

FIG. 5 is a schematic view illustrating the chest strap of FIG. 1 in use,

FIG. 5A is an enlarged view showing the chest strap of FIG. 1 in combination with an ECG signal processor,

FIG. 6 is a plan view of a first embodiment of an ECG electrode covered with an insulator,

FIGS. 6A & 6B are respectively plan and side views of the electrode alone,

FIG. 6C is a schematic side view illustrating the electrode of FIG. 6 connected with an output terminal,

FIG. 7 shows a second embodiment of an ECG electrode, and

FIG. 7A shows a cross-sectional view of the electrode of FIG. 7.

### DETAILED DESCRIPTION OF EXEMPLARY EMBODIMENTS

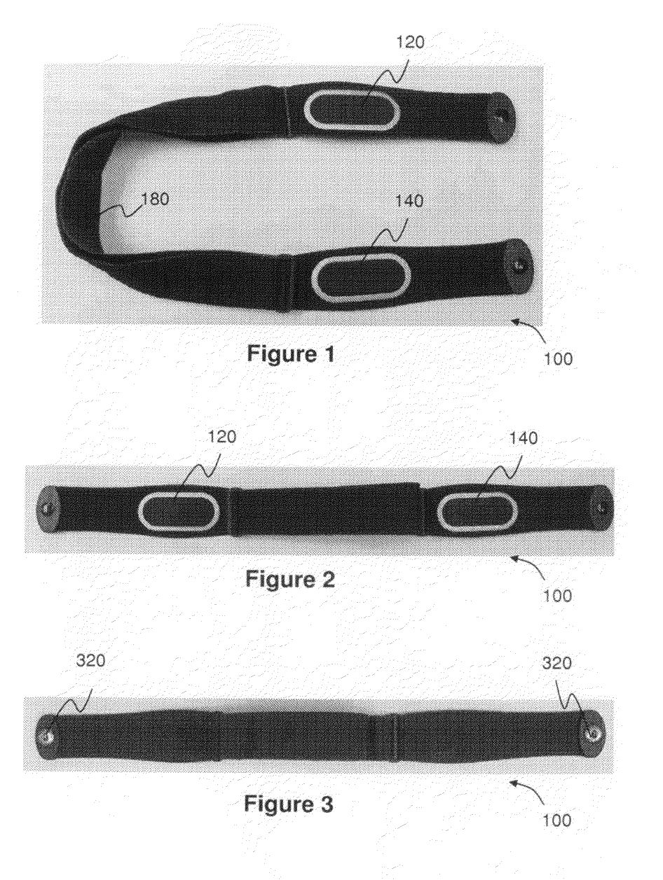

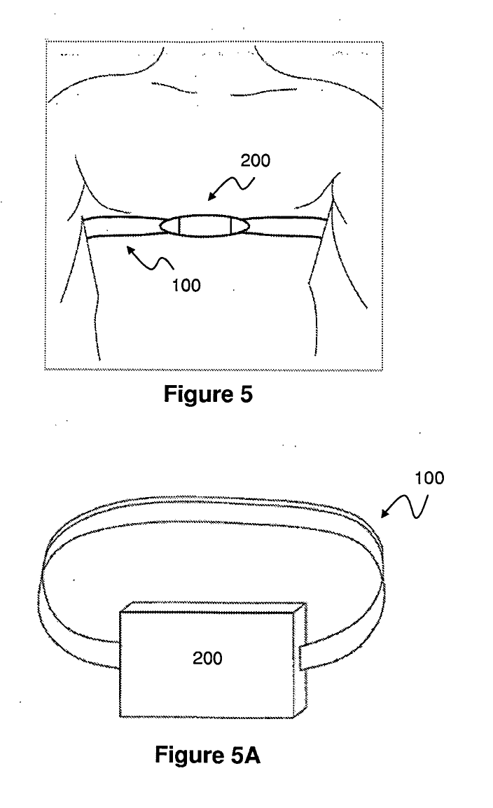

The strap of FIGS. 1 to 5A is a chest-worn ECG signal collection strap apparatus (more commonly known to as a ‘chest strap’) illustrating an exemplary embodiment of a physiological signal collection strap of the present invention. The chest strap 100 comprises first 120 and second 140 ECG signal collection electrodes which are mounted on an elastic strap 180. The elastic strap is made of a breathable and insulating material, such as a fabric comprising a mixture of polyamide and an elastomer. Each of the signal collection electrodes 120, 140 comprises a signal collection pad and a signal output portion. Each signal collection pad comprises a flexible signal collection surface which is arranged to contact the skin and collect ECG signals from a user during use.

A carbonized rubber pad as an example of a flexible signal collection pad is used to collect ECG signals from the chest of a user. The flexible signal collection pad is arranged such that it is urged and pressed against the skin of a user by the tension, more particularly the body-bound tension, of the elastic strap during use when the elastic strap is tightened on the body of the user to minimize contact resistance between the skin and the signal collection pad. The flexible collection surface are arranged to transmit collected ECG signals to a signal output portion which is terminated at a conductive fastening component located at a longitudinal end of the strap. The conductive fastening components of the two ECG signal collection electrodes are located at opposite longitudinal ends of the strap and are arranged to convert the longitudinal strap into a strap loop when the fastening components are respectively anchored onto corresponding or counterpart fastening components. Each conductive fastening component comprises a mechanical mating portion which is adapted for making counterpart engagement with another compatible mechanical mating portion of a compatible fastener. Example of suitable fasteners includes a snap fit fastener or a magnetic clasp.

Each of the signal collection electrodes is mounted on the insulating fabric strap with the signal output portion located at or proximal to a free longitudinal end of the strap such that the two signal output portions are located at the two extreme or free longitudinal ends before the chest strap is converted into a chest strap loop. In this condition, the two signal collection pads are located intermediate the two conductive fasteners which are located at the free longitudinal ends of the strap. To provide good mechanical fastening and good electrical contact, the signal output portion of the signal collection electrode is riveted onto the chest strap by a rigid conductive fastener, which conductive fastener also operates as a signal output terminal to deliver ECG signals from the chest strap for further processing.

In order that ECG signals can be effectively collected from the chest of a user, the signal collection pads of the two ECG signal collection electrodes are disposed on the strap such that when the fastening components are mechanically connected with the respective counterpart or corresponding fastening means to form a tensioned strap loop and duly worn and aligned on the chest of a user, the signal collection surfaces will be attached respectively to the left and right portions of the rib cage of the user. To adapt for this application, the distance between the two signal collection surfaces is equal to or approximately equal to the distance between the portions of the rib cage left and right of the sternum when the chest strap loop is duly worn. In order to provide adequate separation between the two signal collection surfaces during use, the signal collection pad of a signal collection electrode is connected to its corresponding signal output portion via a bridging portion which is adapted to provide spatial separation between the signal collection surface and the signal output portion. For good signal continuity, the signal collection pad, the bridging portion and the output portion are integrally moulded of carbonized rubber as an example of a flexible and conductive substance. It will be noted that the moulded flexible electrode is substantially planar for wearing comfort. The chest strap also comprises a strap length adjustment mechanism for adjusting the effective or usable length of the chest strap to cater for users of different chest widths.

Construction of an exemplary chest strap will be described in more detail below. The elastic strap comprises first and second elongate fabric layer strips which are glued together to form a double-layered strap. Each of the fabric layers are made of an insulating and elastic fabric, such as a mixture of polyamide and elastomer. A breathable fabric which is moisture permeable is preferred for comfort wearing. The planar electrode is sandwiched or embedded between the two fabric layers, while leaving the signal collection surface and the signal output portion exposed for external electrical contact. The regions of the unexposed planar electrode are also glued to the elastic strap for durability. A bonding agent is applied to the periphery of the exposed signal collection surface to further bond the signal collection pad to the strap and to provide a smooth edge. As an optional feature, the back surface of the elastic strap, which is the surface adapted to be in contact with the skin of a user, is formed with a woven anti-slid texture. As a further option, the material composition of the two strap layers could be different. For example, the textured layer may be made of 37% elastomer while the non-textured layer comprises less than 20% elastomer.

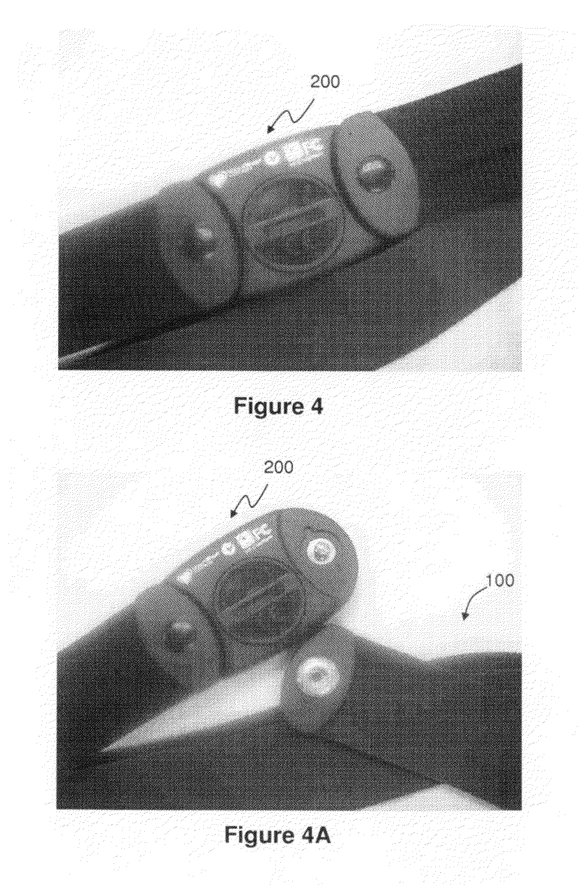

In an exemplary use of the chest strap as shown in FIGS. 4, 4A & 5, the chest strap is illustrated when used in combination with a mobile ECG signal processor 200. The ECG signal processor includes a rigid housing and a pair of signal input terminals. Each signal input terminal is a fastening component having a rigid mechanical mating portion which is configured to engage with a corresponding fastening component of the chest strap. The distance between the two signal collection surfaces in this application configuration therefore includes the longitudinal separation between the two fastening components on the mobile ECG signal processor. The ECG signal processor includes means for capturing and processing the detected ECG signals, as well as optional features for transmitting the relevant data to an external device for further processing or storage.

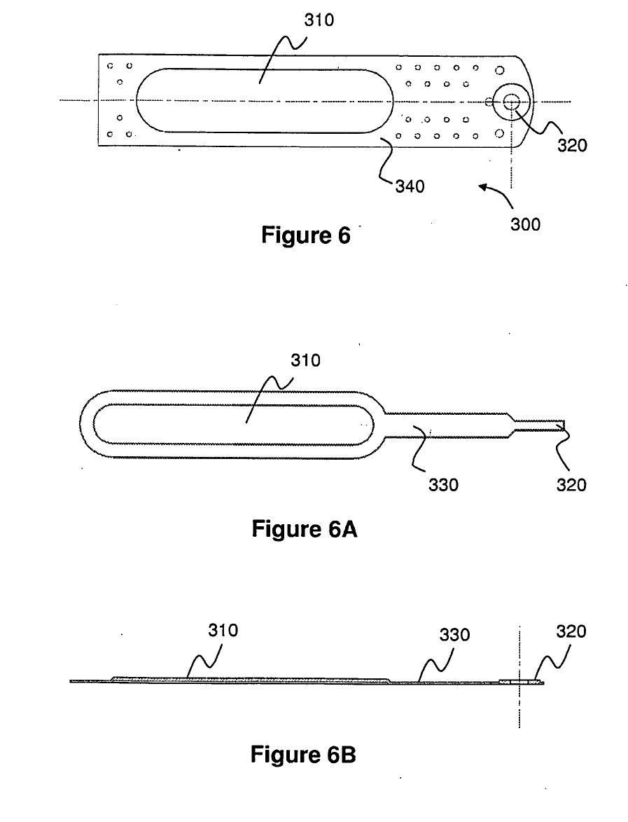

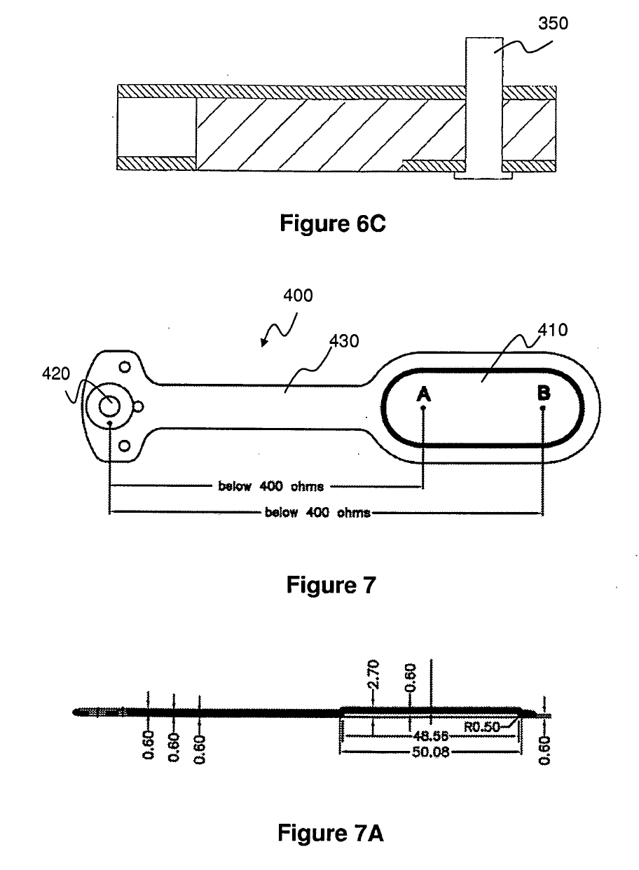

Referring to FIGS. 6 to 6C, there is shown an embodiment of a signal collection electrode 300 comprising a flexible contact pad 310, a signal output portion 320 and a bridging portion 330 interconnecting the flexible contact pad and the signal output portion. The signal collection electrode is integrally moulded of carbonized rubber and is substantially planar and elongate. As is better seen in FIGS. 6B and 6C, the flexible contact portion 310 protrudes above the bridging portion to form an elevated signal collection surface. The entire electrode, except for the signal collection surface and the signal output portion, is covered by an insulator, which is for example a moulded rubber sleeve 340. The rubber sleeve 340 comprises optionally perforations to improve permeability. A mechanical fastening component, for example a snap fastener part, is formed through the signal output portion to form the signal output terminal 350.

The electrode 400 of FIGS. 7 & 7A also comprises a flexible contact pad 410, a signal output portion 420 and a bridging portion 430 and is integrally moulded of a flexible and resilient conductive substance. The bridging portions of the electrodes of FIGS. 6 & 7 are narrower than the width of the signal collection surface. Such a narrowed bridging portion of a resilient material provides additional elasticity to strength the chest strap. In addition, the narrowed bridging portion also eliminates the need of extra breathing perforations.

As an alternative, the bridging portions of the electrodes could be of a same or comparable width to that of the signal collection surface, and perforations may be optionally formed outside the signal collection surfaces to improve breathability.

Regardless of the specific electrode designs, each of the signal collection pad is configured so that the effective area of the distributed signal collection surface could collect reasonable ECG signals for meaningful processing. The effective area here refers to the surface which exposes through the fabric strap and which has a typical width of about 1.5 to 2.5 cm and a typical length of about 5-8 cm. The resistance of the electrode, measured between the signal collection pad and the signal output portion, is typically below 1 kΩ for good signal processing and operation.

In an exemplary use of the chest strap for swim applications, the electrode resistance is typically below 1,000Ω, preferably below 700Ω, and more preferably below 500Ω and even 400Ω. It is noted that such a low resistance provides a useful solution to enable ECG signal collection in both sea and chlorinated water. When using as a swim chest strap, the bridging portion is insulated by a moulded collar which is mould over the electrode, leaving only the signal collection surface and the signal output portion exposed. The insulated bridging portion mitigates signal leakage or interference when the chest is used in a highly conductive medium such as sea or chlorinated water.

While the present invention has been explained with reference to the embodiments above, it should be appreciated that the embodiments are only described to illustrate the invention and are not meant for restricting the scope of invention. For example while a swim monitor strap has been illustrated as an example, a strap of the present invention could be used for running or other activities without loss of generality.

Table of Numerals

| | | |
|---|---|---|
| 100 | | Chest strap |
| | 120 | First ECG signal collection electrode |
| | 140 | Second ECG signal collection electrode |
| | 180 | Strap |
| 200 | | Mobile ECG signal processor |
| 300 | | Signal collection electrode |
| | 310 | Contact pad |
| | 320 | Signal output portion |
| | 330 | Bridging portion |
| | 340 | Sleeve |
| | 350 | Signal output terminal |
| 400 | | Electrode |
| | 410 | Contact pad |
| | 420 | Output portion |
| | 430 | Bridging portion |

1.-36. (canceled)

37. A physiological signal collection electrode comprising:
a signal collection pad;
a signal output pad; and
an elongate bridging portion interconnecting the signal collection pad and the signal output pad,
wherein the signal collection pad comprises a skin contact portion and defines a skin contact surface;
wherein the signal collection pad, the signal output portion and the bridging portion are integrally molded of a flexible, conductive and resilient material; and
wherein the bridging portion is insulated by an insulating material which is flexible, resilient and waterproof.

38. A physiological signal collection electrode according to claim 37, wherein the insulating material is over-molded onto the bridging portion.

39. A physiological signal collection electrode according to claim 37, wherein the flexible, resilient and conductive material is selected form carbonized rubber, carbonized fabrics, Nickel copper plated polyester, silver nylon mesh, cotton silver bamboo fiber, or other conductive fabrics such as metalized fabrics or the like.

40. A physiological signal collection electrode according to claim 37, wherein the width of the bridging portion is substantially smaller than that of the skin contact portion.

41. A physiological signal collection electrode according to claim 37, wherein the signal output pad includes a signal output terminal, and wherein the effective resistance of the electrode between the skin contact portion and the signal output terminal is less than 1 kΩ, preferably less than 500Ω, and more preferably less than 400Ω.

42. A physiological signal collection electrode according to claim 37, wherein the skin contact portion is flexible and elongate with an effective skin contact surface of between 7.5 cm² to 20 cm², wherein the skin contact portion is between 1.5 -2.5 cm wide and 5-8 cm long, and wherein the narrowed bridging portion is between 6 and 10 cm long and about half or less than half of the width of the skin contact portion.

43. An ECG signal collection strap comprising:
a first ECG signal collection electrode and a second ECG signal collection electrode which are respectively mounted at first and second free longitudinal ends of an insulating strap;
wherein each ECG signal collection electrode comprises:
a signal collection pad;
a signal output pad; and
an elongate bridging portion interconnecting the signal collection pad and the signal output pad,
wherein the signal collection pad comprises a skin contact portion and defines a skin contact surface;
wherein the signal collection pad, the signal output portion and the bridging portion are integrally molded of a flexible, conductive and resilient material; and
wherein the bridging portion is insulated by an insulating material which is flexible, resilient and waterproof.

44. An ECG signal collection strap according to claim 43, wherein the insulating material is over-molded onto the bridging portion.

45. An ECG signal collection strap according to claim 43, wherein the signal output pad includes a signal output terminal, and wherein the effective resistance of the electrode between the skin contact portion and the signal output terminal is less than 1 kΩ, preferably less than 500Ω, and more preferably less than 400Ω.

46. An ECG signal collection strap according to claim 43, wherein first and second mechanical fastening parts adapted for converting the longitudinal strap into a strap loop for wearing on the body of a user are respectively mounted at the first and second longitudinal ends of the insulating strap, the first and second mechanical fastening parts being arranged such that tension in the strap will urge the skin contact surfaces of the first and second electrodes to abut against skin of the user to make electrical contacts and to collect physiological signals from the user when the strap loop is worn on the body of the user with the fastening parts engaged; and wherein each of the mechanical fastening parts forms a signal output terminal of its associated ECG signal collection electrode,

47. An ECG signal collection strap according to claim 46, wherein the first and second mechanical fastening parts are adapted for making electrical and mechanical engagement with a portable ECG signal processing device whereby a complete strap loop is formed upon the making of the mechanical engagement and ECG signals collected by the electrodes are delivered to the portable ECG signal processing device via the mechanical fastening parts.

48. An ECG signal collection strap according to claim 47, wherein the first and second mechanical fastening parts are complementary mating portions of a snap fastener, and wherein the mechanical fastening parts and the signal collection pad are oppositely facing on the insulating strap.

49. An ECG signal collection strap according to claim 46, wherein the signal collection pads of the first and second ECG signal collection electrodes are adapted for making signal collection contact with the left and right rib cage portions of a user when the strap is worn with the first and second mechanical fastening parts engaged; wherein the insulating strap is length adjustable and made of an air and/or moisture permeable elastomeric fabric such as elastomeric polyamide or swimwear fabric such as Nylon, Lycra®, polyester, or the like; and wherein an anti-slid texture is formed on portions of the insulating strap which are in contact with user skin during use.

50. An ECG signal collection strap according to claim 43, wherein the bridging portion of each of the electrodes is embedded within the insulating strap and the signal collection pad of each of the electrodes is bonded on the insulating strap.

51. An ECG signal collection strap according to claim 43, wherein the insulating strap is an elastic tensioning strap, and the signal output terminals of the first and second ECG signal collection electrodes are the end terminals of the tensioning strap; and wherein the strap loop is a body strap such as a chest strap, head strap, arm strap, thigh strap, or the like.

52. A swim performance monitor comprising:
a swim motion detector adapted to detect swim motion data;
an ECG signal processing device adapted to process physiological data of a user; and
an ECG signal collection strap,
wherein the ECG signal collection strap comprises a first ECG signal collection electrode and a second ECG signal collection electrode which are respectively mounted at first and second free longitudinal ends of an insulating strap;
wherein each ECG signal collection electrode comprises a signal collection pad, a signal output pad, and an elongate bridging portion interconnecting the signal collection pad and the signal output pad;
wherein the signal collection pad comprises a skin contact portion and defines a skin contact surface;
wherein the signal collection pad, the signal output portion and the bridging portion are integrally molded of a flexible, conductive and resilient material;
wherein the bridging portion is insulated by an insulating material which is flexible, resilient and waterproof;
wherein the swim motion detector and the ECG signal processing device are formed as a module and are housed within a common housing, and the ECG signal processing device has first and second signal input terminals; and
wherein the ECG signal processing device is arranged to obtain ECG signals from the ECG signal collection strap via its signal output terminals.

53. A swim performance monitor according to claim 52, wherein the swim motion detector comprises a 3-axis accelerometer which is arranged to collect swim motion data.

54. A swim performance monitor according to claim 52, wherein the effective signal path resistance between the skin contact portion of the electrode and a corresponding output terminal is less than that of the swim water signal path between the skin in contact with the electrode and the corresponding output terminal.

55. A swim performance monitor according to claim 52, wherein the monitor is adapted for use in sea water, chlorinated water or other ionized water, and the effective signal path resistance of each of the electrodes is below 800 ohms, preferably below 500 ohms.

56. A swim performance monitor according to claim 52, wherein each of the first and second terminals of the ECG processing device is formed as a mating portion of a mechanical fastener part, and each of the output terminals of the strap is formed into a complementary mating portion of a counterpart mechanical fastener part.
